(19) United States
(12) Patent Application Publication
(10) Pub. No.: US 2011/0030700 A1
(43) Pub. Date: Feb. 10, 2011

(54) Snoring Treatment and Associated Apparatus, System and Method

(76) Inventor: John K. Wilson, Wilton, CT (US)

Correspondence Address: MCCARTER & ENGLISH, LLP STAMFORD, CANTERBURY GREEN, 201 BROAD STREET, 9TH FLOOR, STAMFORD, CT, 06901, US

(21) Appl. No.: 12/856,949

(22) Filed: Aug. 16, 2010

Related U.S. Application Data
(63) Continuation-in-part of application No. 11/512,542, filed on Aug. 30, 2006, now Pat. No. 7,775,211.

(51) Int. Cl. A61F 5/56

(52) U.S. Cl. 128/848

(57) ABSTRACT

Apparatus, systems and methods effective in treating snoring behavior are provided. The treatment apparatus include a mouthpiece, a first chamber in communication with the mouthpiece, a displaceable member mounted with respect to the first chamber for applying a resistive force as gas is withdrawn from the first chamber, and an independent source of negative pressure acting upon the displaceable member. The evacuation force is increased due to the force required to displace the displaceable member positioned within the disclosed treatment apparatus. The independent source of negative pressure, e.g., a vacuum pump, increases the resistive force applied by the displaceable member. The displaceable member may take the form of a flexible membrane, piston or diaphragm that is mounted with respect to the inner wall of the first chamber or the second chamber. Use of the treatment apparatus is effective in reducing and/or eliminating snoring behavior for extended periods.

### BACKGROUND

1. Cross Reference to Related Application

The present application is a continuation-in-part application that claims priority benefit to a co-pending non-provisional patent application entitled “Snoring Treatment and Associated Apparatus, System and Method,” which was filed on Aug. 30, 2006, and assigned Ser. No. 11/512,542. The entire content of the foregoing non-provisional patent application is incorporated herein by reference.

2. Technical Field

The present disclosure is directed to apparatus, systems and methods for snoring treatment and, more particularly, to an apparatus and treatment regimen that provides relief from snoring through utilization of a therapeutic device that ameliorates, in whole or in part, the condition.

3. Background Art

Approximately one third of the U.S. population, more than 80 million people, is reported to snore on a regular basis (Mayo Clinic). Snoring deprives both the individual who is snoring and any sleeping companions of sleep. Indeed, the Mayo Clinic reports that each of the noted individuals loses one hour of sleep per night due to such snoring behavior, with corresponding negative impacts on health and quality of life. More ominously, as many as twenty four percent (24%) of Americans are reported to suffer from sleep-disordered breathing (Scientific American, Jun. 6, 2005). Individuals suffering from obstructive sleep apnea have been found to stop breathing for up to fifteen (15) seconds with a remarkable frequency, e.g., hundreds of times per night. This condition increases the risk and/or incidence of high blood pressure, heart attack and stroke. According to the New England Journal of Medicine (Mar. 24, 2005), individuals suffering from obstructive sleep apnea are three (3) times as likely to die from a heart attack during the night. In addition, snoring generally increases in direct relation to age and weight, demographics that are evidencing significant increases in today's population.

Available treatment options for snoring generally fall into three categories: (1) highly invasive surgery (e.g., laser-assisted uvula palatoplasty (LAUP), and radio frequency surgical ablation (RFTA)), (2) relatively invasive, uncomfortable consumer devices (e.g., masks) with unproven effectiveness, and (3) over-the-counter remedies (e.g., nasal strips and throat sprays) with limited effectiveness. In view of the significant implications associated with snoring that remains untreated, effective treatment systems, methods and modalities are needed. In particular, effective treatment systems that are easy-to-use, non-invasive and with proven efficacy are needed.

The apparatus, systems and methods of the present disclosure address such needs, serving to reduce and/or eliminate snoring through non-invasive and easy-to-use treatment regimens, as will be apparent from the description which follows.

### SUMMARY

An advantageous apparatus, system and method is provided for snoring treatment and reducing/eliminating the incidence of snoring by a user of the apparatus, system and/or method. According to exemplary embodiments hereof, the apparatus includes a mouthpiece, a first chamber in communication with the mouthpiece, and a displaceable member mounted with respect to the first chamber for applying a resistive force as gas is withdrawn from the first chamber. A second chamber is advantageously defined on the opposite side of the displaceable member, such second chamber generally defining a substantially airtight region. Based on the positioning of the displaceable member between the first and second chambers, the evacuation force required to deflect, deform or otherwise displace the displaceable member is increased.

In exemplary embodiments of the disclosed apparatus, the displaceable member may take the form of a flexible membrane, piston or diaphragm that is mounted with respect to the inner wall of the first chamber or the second chamber. In either case, the displaceable member responds to a user's attempted withdrawal of air from the first chamber, i.e., by sucking air through the mouthpiece, by deflecting, deforming or otherwise displacing toward the mouthpiece. The resistance experienced by the user generally increases as air is withdrawn/evacuated from the first chamber based on at least one of the following factors: (1) increased resistance offered by the displaceable member as it is asked to deflect, deform or otherwise displace to a greater extent, (2) the resistance of the air within a substantially airtight second region to expanding into the additional volume defined by the deflection, deformation or other displacement of the displaceable member toward the mouthpiece, and/or (3) an auxiliary source of resistive force, e.g., a vacuum pump or other source of negative pressure in fluid communication with the second region.

An indicator mechanism may be mounted with respect to the displaceable member to provide a visually perceptible indication of the user's interaction with the disclosed apparatus, e.g., the degree to which the displaceable member is displaced toward the mouthpiece based on the user's interaction therewith. In an exemplary embodiment, the indicator mechanism includes a linkage assembly that is fixed with respect to the displaceable member and that translates travel of the displaceable member relative to the first/second chambers into a visually perceptible indication. Thus, a slot or window may be provided in a wall of the second chamber through which an indicator may be visible, such indicator traveling relative to the slot/window based upon and in response to travel of the displaceable member relative to the first/second chambers. In exemplary embodiments of the present disclosure, the slot/window is sealed so as to maintain an air-tight seal within the second region of the disclosed apparatus.

According to exemplary embodiments of the present disclosure, a system for snoring treatment is provided that includes, inter alia, an apparatus and a source of negative pressure, e.g., a vacuum pump, adapted for fluid communication with the apparatus. The apparatus generally includes a mouthpiece, a first chamber in communication with the mouthpiece, a displaceable member mounted with respect to the first chamber, and a second chamber opposite the first chamber relative to the displaceable member. Of note, the source of negative pressure is generally adapted for fluid communication with the second chamber. In exemplary embodiments, the source of negative pressure may be detachably connected relative to the second chamber, e.g., through a threaded fitting, a snap-connect or the like. In other implementations, the source of negative pressure may be permanently and/or integrally associated with the second chamber.

The present disclosure further provides an advantageous treatment regimen for reducing and/or eliminating a user's snoring. According to exemplary embodiments of the disclosed treatment regimen, an individual is provided with an apparatus/device that includes a mouthpiece, a first chamber in communication with the mouthpiece, and a displaceable member mounted with respect to the first chamber for applying a resistive force with respect to the flow of gas from the first chamber, and (ii) withdrawing air from the first chamber through the mouthpiece against the resistance applied by the displaceable member. In further exemplary embodiments, an ancillary source of negative pressure is associated with a second chamber positioned opposite the first chamber, such source of negative pressure delivering greater resistance to air withdrawal from the first chamber. To gain clinically significant results from the disclosed method, the user is generally required to perform the noted “air withdrawal” step several times prior to retiring to bed. Between each repetition, the displaceable member advantageously and automatically returns to its initial rest position in preparation for the next repetition. The source of negative pressure may be “turned off” between repetitions, and then reenergized during each successive repetition in use. In an exemplary embodiment of the disclosed treatment regimen, the user performs the air withdrawal step for a period of between one second and fifteen seconds. Generally, favorable results may be expected when the treatment regimen is performed within one hour of the individual retiring to bed.

While not desiring to be limited to a particular clinical and/or anatomical explanation as to the reasons for the effectiveness of the disclosed snoring treatment regimen, it is believed that the advantageous snoring reduction/elimination is effectuated through the lifting of a user's palette based on use of the disclosed apparatus. Indeed, it is believed that lifting of a user's palette to a requisite degree is effective in reducing/eliminating a user's snoring, and that repeated treatments over a period of time may be effective in permanently or semi-permanently lifting a palette such that snoring is reduced/eliminated for an extended period, e.g., for a period of days, weeks, months, etc. Preliminary tests with the disclosed apparatus and associated treatment regimen have demonstrated snoring-free sleep patterns for extended periods, e.g., about six hours.

Additional advantageous features, functions and benefits of the disclosed apparatus, system and treatment methods will be apparent from the detailed description which follows, particularly when read in conjunction with the appended figures.

### BRIEF DESCRIPTION OF THE FIGURES

To assist those of ordinary skill in the art in making and using the disclosed apparatus/systems, reference is made to the accompanying figures, wherein:

FIG. 1 is a front view of an exemplary treatment apparatus according to the present disclosure;

FIG. 2 is a cross-sectional view of the exemplary treatment apparatus of FIG. 1;

FIG. 3 is a view of an exemplary mouthpiece component according to the disclosed treatment apparatus;

FIG. 4 is a plan view of an exemplary outer housing component according to an exemplary embodiment of the present disclosure;

FIG. 5 is a plan view of an intermediate housing component that is adapted to receive a displaceable member according to the present disclosure;

FIG. 6 is a plot of improved snoring behavior achieved through use of the disclosed treatment apparatus;

FIG. 7 is a plan view of an alternative apparatus according to the present disclosure that includes a second chamber that does not define an air-tight seal;

FIG. 8 is a plan view of a system according to the present disclosure that includes a treatment apparatus and associated source of negative pressure.

### DESCRIPTION OF EXEMPLARY EMBODIMENT(S)

The disclosed apparatus, systems and methods are effective in snoring treatment. As described in greater detail below, exemplary embodiments of the disclosed treatment apparatus include a mouthpiece, a first chamber in communication with the mouthpiece, and a displaceable member mounted with respect to the first chamber for applying a resistive force as gas is withdrawn from the first chamber. A second, substantially airtight chamber is generally defined on the opposite side of the displaceable member. A source of negative pressure, e.g., a vacuum pump, may be associated with the second chamber to deliver a further resistive force to the disclosed treatment apparatus. The evacuation force required to deflect, deform or otherwise displace the displaceable member is increased due to the force required to displace the displaceable member positioned within the disclosed treatment apparatus. In exemplary embodiments of the disclosed apparatus, the displaceable member may take the form of a flexible membrane, piston or diaphragm that is mounted with respect to the inner wall of the first chamber or the second chamber.

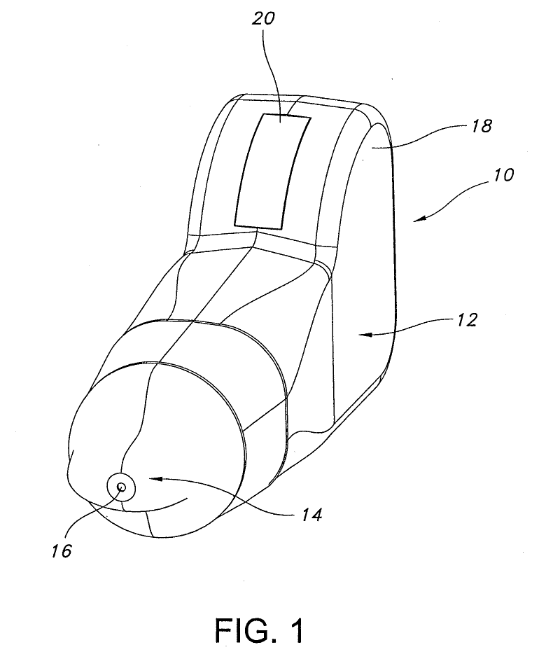

Turning to FIG. 1, an exemplary treatment apparatus 10 is depicted. Treatment apparatus 10 includes an outer housing 12 and a mouthpiece 14. The mouthpiece 14 is generally configured and dimensioned to be comfortably positioned within a user's mouth. Chamfered and/or radiused surfaces may be provided to enhance user interaction therewith. An opening 16 is formed in mouthpiece 14 to permit air passage therethrough. As used herein, the term “air” includes both respirated and non-respirated air. While the overall size/dimensional characteristics of outer housing 12 are not critical, the outer housing is generally of sufficient size to define internal chambers that are effective to achieve the snoring treatment effects described herein. According to exemplary embodiments of the present disclosure, the outer housing 12 is also sized so as to be easily held/handled by a user of the treatment apparatus. Exemplary dimensions of an illustrative treatment apparatus include a length (from mouthpiece to end of second chamber) of about 7 inches, a width of about 1.5 to 2 inches, and a height in the bulbous region 18 (described below) of about 3 inches. Alternative geometries may be employed without departing from the spirit or scope of the present disclosure.

With further reference to FIG. 1, outer housing 12 defines a distal bulbous region 18. A sealed slot or window 20 is formed on the face of bulbous region 18. Of note, due to the upward expanse of bulbous region 18 relative to mouthpiece 14, slot/window 20 is substantially in visual alignment with a user's eyes when he/she positions the mouthpiece 14 in his/her mouth. As described in greater detail herein below, slot/window 20 cooperates with a visual indicator mechanism that reflects the user's interaction with the treatment apparatus.

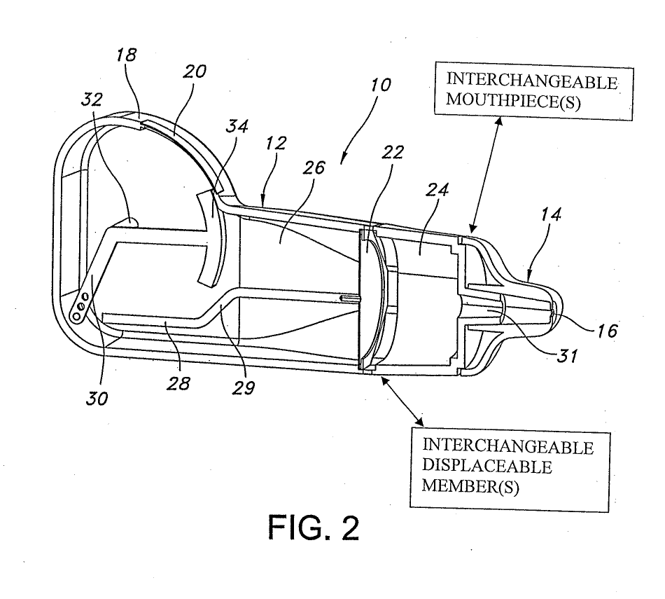

Turning to FIG. 2, a cross-sectional view of treatment apparatus 10 is provided. A displaceable member 22 is positioned within and mounted with respect to outer housing 12. In the exemplary embodiment of FIG. 2, displaceable member 22 takes the form of an elastic or flexible membrane fabricated from a rubber or rubber-like material. However, the displaceable member 22 may take various alternative forms, e.g., a piston assembly that is adapted to translate relative to the outer housing 12 in response to forces as described herein. Thus, the present disclosure expressly encompasses alternative displaceable member designs, as will be readily apparent to persons skilled in the art based on the disclosure provided herein.

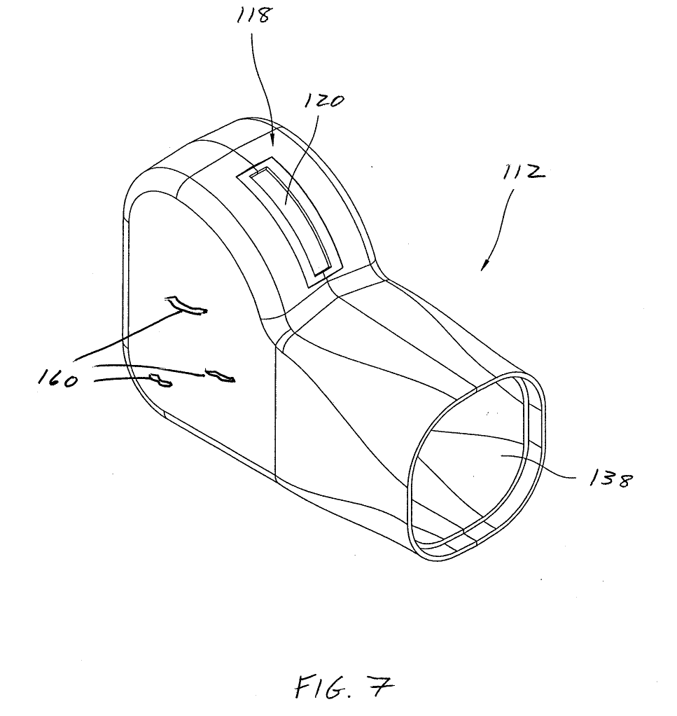

A first chamber 24 is defined on the mouthpiece side of displaceable member 22 and a second chamber 26 is formed on the opposite side thereof. The second chamber 26 extends into bulbous region 18 and is generally airtight, i.e., air captured therewithin cannot escape from outer housing 12. In alternative embodiments (see FIG. 7), the second chamber is not substantially airtight. First chamber 24 communicates through mouthpiece 14 and opening 16 to the external atmosphere. Of note, a channel 31 is formed within mouthpiece 14 to funnel/guide air between opening 16 and first chamber 24. The channel 32 may take the form of a cylindrical extension positioned within mouthpiece 14.

A first linkage 28 is mounted with respect to displaceable member 22, e.g., through an adhesion technique. First linkage 28 is mounted with respect to a second linkage 30 (although in FIG. 2 the two linkages 28, 30 are separated for clarity purposes). First linkage 28 defines a jog 29 that facilitates operation of the disclosed linkage mechanism. Second linkage 28 defines an angled structure, typically an obtuse angle, and is pivotally mounted with respect to outer housing 12, e.g., with respect to a pair of mounting extensions 32 (only one mounting extension 32 is visible in the cross-sectional view of FIG. 2) that extend inwardly from opposed faces of the outer housing 12. An arcuate indicator surface 34 is formed or mounted to the end of second linkage 30. Indicator surface 34 is adapted to cooperate with sealed slot/window 20 formed in outer housing 12 to indicate travel/displacement of the displaceable member 22 relative to the outer housing 12.

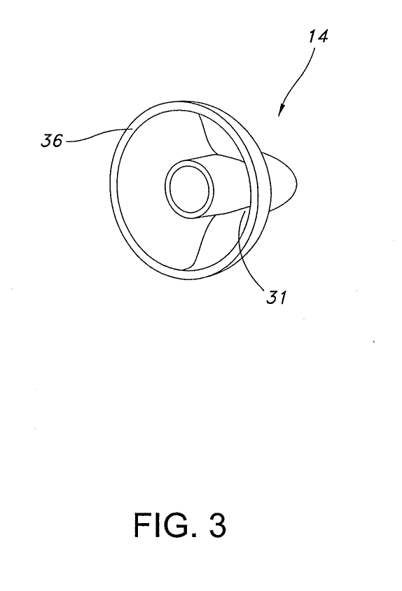

Additional details of an exemplary mouthpiece 14 according to the disclosed exemplary treatment apparatus are depicted in FIG. 3. As shown therein, a cylindrical channel 31 is defined within mouthpiece 14 for guiding air to and from mouthpiece opening 16 (not visible). In the exemplary mouthpiece 14 depicted in FIG. 3, a substantially circular mounting face 36 is defined thereby. Mounting face 36 facilitates assembly of mouthpiece 14 to the other component(s) associated with the disclosed treatment apparatus 10. Thus, for example, mouthpiece 14 may be designed to be detachable/replaceable from the other component(s), thereby permitting multiple users to have personal mouthpieces for use therewith. In such circumstances, a “quick-connect” feature may be incorporated into/onto mouthpiece 14, e.g., a screw thread, snap-lock, bayonet lock or the like.

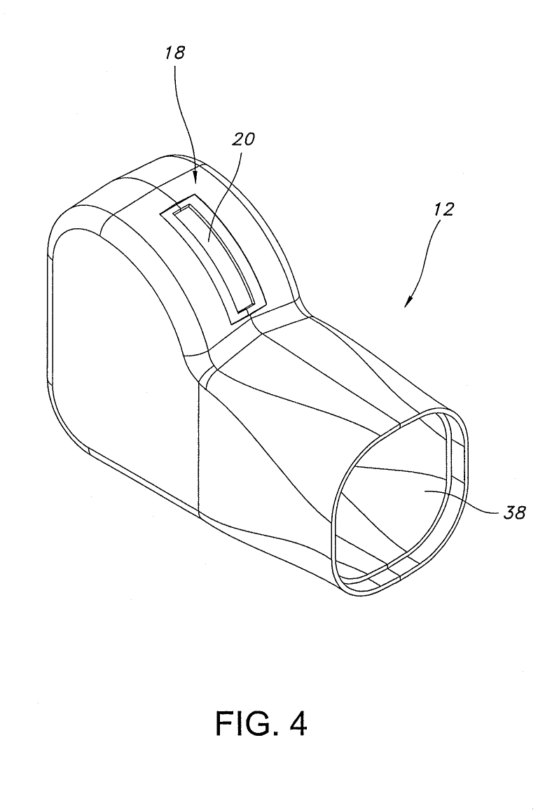

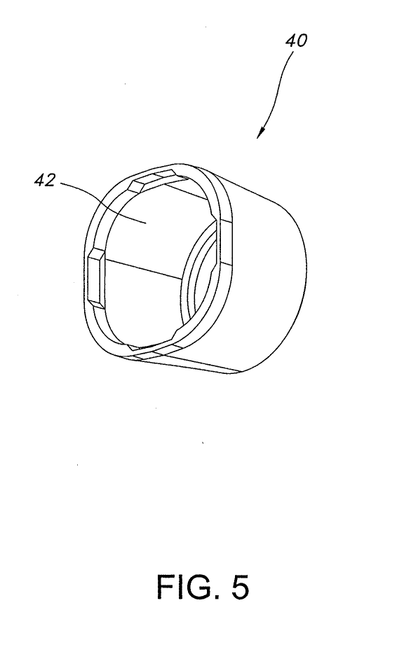

FIG. 4 provides a further view of a portion of an exemplary outer housing 12 according to the present disclosure. An opening 38 is defined in a forward region thereof for cooperation with an intermediate component 40, as shown in FIG. 5. The displaceable member 22 is typically mounted with respect to the opening 42 defined in intermediate component 40, although alternative mounting designs may be employed without departing from the spirit or scope of the present disclosure. Once a displaceable member 22 is mounted with respect to the intermediate component 40, the intermediate component and the portion of the outer housing 12 shown in FIG. 4 (with the linkage mechanism extending into the internal cavity of such outer housing portion in the manner shown in FIG. 2), the mouthpiece 14 may be secured with respect thereto, thereby forming a complete treatment apparatus assembly.

In typical use of treatment apparatus 10, the displaceable member 22 responds to a user's attempted withdrawal of air from the first chamber 24, i.e., by sucking air through the opening 16 in the mouthpiece 12, by deflecting, deforming or otherwise displacing toward the mouthpiece 14. The resistance experienced by the user generally increases as air is withdrawn/evacuated from the first chamber 24 based on at least one of the following factors: (1) increased resistance offered by the displaceable member 22 as it is asked to deflect, deform or other displace to a greater extent, and/or (2) the resistance of the air within the substantially airtight second chamber 26 to expanding into the additional volume defined by the deflection, deformation or other displacement of the displaceable member 22 toward the mouthpiece 14.

An indicator mechanism is defined by the linkage mechanism described herein, i.e., linkages 28, 30 and indicator surface 34 may be mounted with respect to the displaceable member 22 to provide a visually perceptible indication of the user's interaction with the disclosed treatment apparatus 10, e.g., the degree to which the displaceable member 22 is displaced toward the mouthpiece 14 based on the user's interaction therewith. The linkage assembly is fixed with respect to the displaceable member 22 and translates travel of the displaceable member 22 relative to the first/second chambers 24, 26 into a visually perceptible indication. Thus, the slot or window 20—which may be sealed—permits viewing of indicator surface 34 therethrough, such indicator surface 34 traveling relative to the slot/window 20 based upon and in response to travel of the displaceable member 22 relative to the first/second chambers 24, 26.

An advantageous treatment regimen for reducing and/or eliminating a user's snoring is provided through use of the disclosed treatment apparatus. According to exemplary embodiments of the disclosed treatment regimen, an individual is provided with an apparatus/device that includes a mouthpiece, a first chamber in communication with the mouthpiece, and a displaceable member mounted with respect to the first chamber for applying a resistive force with respect to the flow of gas from the first chamber, and such user withdraws air from the first chamber through the mouthpiece against the resistance applied by the displaceable member. To gain clinically significant results from the disclosed method, the user is generally required to perform the noted “air withdrawal” step several times prior to retiring to bed. Between each repetition, the displaceable member advantageously and automatically returns to its initial rest position in preparation for the next repetition. In an exemplary embodiment of the disclosed treatment regimen, the user performs the air withdrawal step for a period of between one minute and fifteen minutes. Generally, favorable results may be expected when the treatment regimen is performed within one hour of the individual retiring to bed.

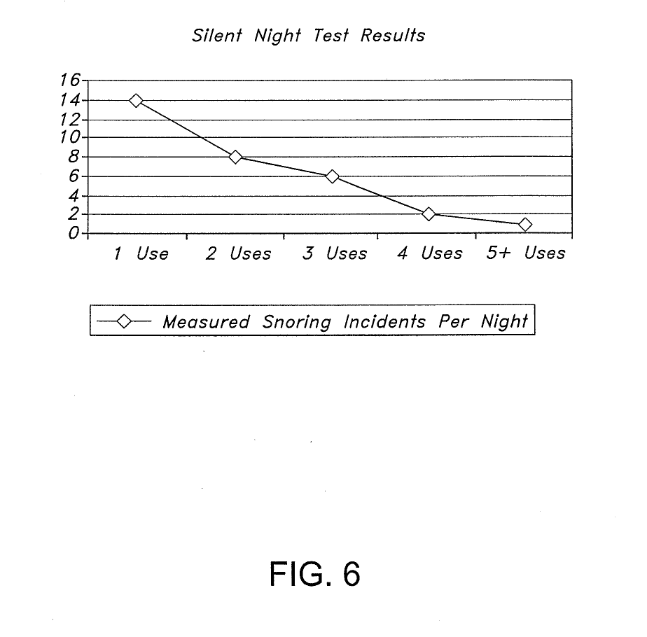

Indeed, FIG. 6 demonstrates extremely favorable, preliminary results achieved through use of the disclosed treatment apparatus on repeated occasions, with snoring incidents (as measured by a sound-actuated recording device) decreasing significantly. In the preliminary results set forth in FIG. 6, snoring incidents were reduced to only fourteen (14) based on one use of the disclosed treatment apparatus, and fell to less than two (2) snoring incidents after five (5) uses.

While not being limited to a particular clinical and/or anatomical explanation in support of the disclosed treatment regimen, it is believed that the advantageous reduction/elimination of snoring is effectuated through the lifting of the user's palette. Indeed, it is believed that lifting of a user's palette is effective to reduce/eliminate snoring in a user, and that repeated treatments over a period of time may be effective in permanently or semi-permanently lifting a palette such that snoring is reduced/eliminated for an extended period, e.g., days, weeks, months, etc.

Turning to FIG. 7, an alternative implementation of the present disclosure is schematically depicted. Outer housing 112 defines an opening 138 in a forward region thereof for cooperation with an intermediate component, e.g., component 40 shown in FIG. 5. As discussed with reference to the previous exemplary embodiment, the displaceable member is typically mounted with respect to an opening defined in intermediate component, although alternative mounting designs may be employed without departing from the spirit or scope of the present disclosure. Once a displaceable member is mounted with respect to the intermediate component, the intermediate component and the portion of the outer housing 112 shown in FIG. 7, a mouthpiece may be secured with respect thereto, thereby forming a complete treatment apparatus assembly. With further reference to FIG. 7, venting openings 160 are defined in outer housing 112 so as to allow entry of ambient air thereto. The design, positioning, size and overall area associated with vent openings 160 may be selected based on the desired level of ambient air introduction. Alternatively, ambient air may be allowed to pass to the interior of outer housing 160 by assembling components in a non-airtight seal manner and/or allowing ambient air to enter through slot/window 120.

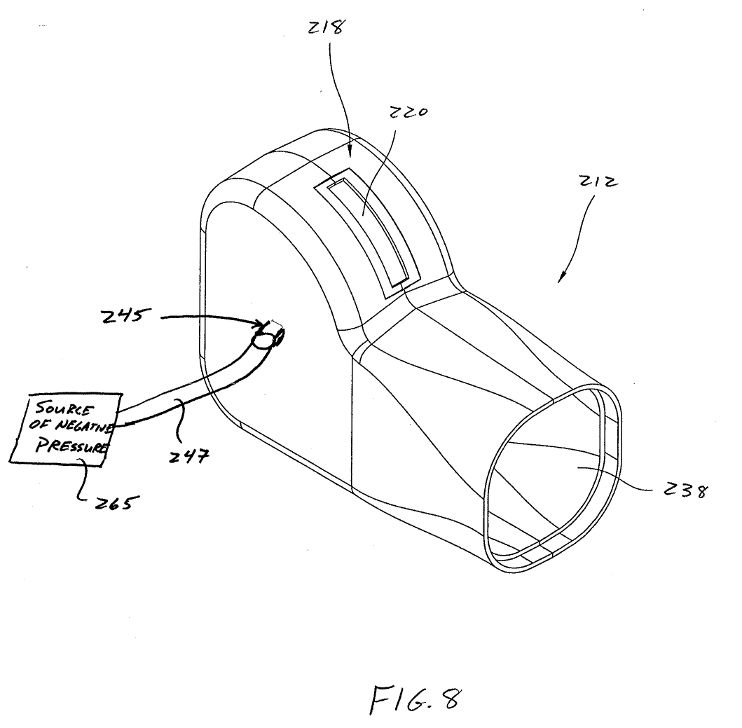

With reference to FIG. 8, a further exemplary implementation of the present disclosure is provided wherein outer housing 212 (which includes comparable features to those described with reference to FIGS. 4 and 7) is adapted for use in conjunction with a source of negative pressure, which is shown schematically as element 265. The negative pressure source 265 may take various forms, e.g., a vacuum pump, a syringe for withdrawal of air from the region within outer housing 212, and the like. Negative pressure source 265 may communicate with the interior of outer housing 212 through conduit 247 or other appropriate fluid communication means. In addition, negative pressure source 265 may be permanently/integrally connected with respect to outer housing 265 or may be detachably fixed relative thereto, e.g., by way of a fitting 245 that is formed in and/or extends from outer housing 265. The negative pressure source 265 may be employed to deliver further resistive force to movement of the displaceable member associated with the disclosed treatment apparatus. The treatment apparatus and negative pressure source may be offered/supplied as a system or kit, as will be readily apparent to persons skilled in the art.

Although the present disclosure has been described with reference to exemplary embodiments of the advantageous treatment apparatus, system and treatment regimen, the present disclosure is not limited by such exemplary embodiments. Rather, such exemplary embodiments are merely illustrative of potential implementations of the disclosed treatment apparatus, system and method. Indeed, the present disclosure expressly encompasses enhancements, modifications and/or variations on the disclosed embodiments that do not depart from either the spirit or scope of the present disclosure.

1. A method for snoring treatment, comprising:
a. providing a treatment apparatus that includes a mouthpiece, a first chamber in communication with the mouthpiece, a displaceable member mounted with respect to the first chamber for applying a resistive force with respect to the flow of gas from the first chamber,
b. providing an independent source of negative pressure that acts upon the displaceable member to increase the resistive force applied thereto; and
c. withdrawing air from the first chamber through the mouthpiece against the resistance applied by the displaceable member and the independent source of negative pressure, thereby causing the indicator mechanism to provide an indication of the relative position of the displaceable member.

2. A method according to claim 1, wherein the treatment apparatus further includes a second chamber defined opposite the first chamber, the second chamber being positioned on an opposite side of the displaceable member relative to the first chamber.

3. A method according to claim 1, wherein the independent source of negative pressure acts upon the displaceable member for a limited period of time

4. A method according to claim 3, wherein the predetermined period of time is between one second and fifteen seconds.

5. A method according to claim 1, wherein the displaceable member is selected from a flexible membrane, a piston, a diaphragm, and combinations thereof.

6. A method according to claim 1, wherein the displaceable member provides resistance to withdrawal of air through the mouthpiece and automatically resumes a non-displaced orientation when air withdrawal is discontinued and air reenters the first chamber through the mouthpiece.

7. A method according to claim 1, wherein air is repeatedly withdrawn from the first chamber by a user prior to retiring to bed.

8. A method according to claim 7, wherein air is repeatedly withdrawn from the first chamber for a period of one second to fifteen seconds.

9. A method according to claim 1, wherein the treatment regimen is effective to ameliorate an individual's snoring.

10. A method according to claim 9, wherein the treatment regimen is effective to prevent snoring for a period of up to six hours.

11. An apparatus for snoring treatment, comprising:
a. a mouthpiece;
b. a first chamber in communication with the mouthpiece;
c. a displaceable member mounted with respect to the first chamber for applying a resistive force with respect to the flow of gas from the first chamber; and
d. an independent source of negative pressure in communication with the displaceable member adapted to act upon the displaceable member to increase the resistive force applied thereby.

12. An apparatus according to claim 11, further including a second chamber defined opposite the first chamber.

13. An apparatus according to claim 12, wherein the independent source of negative pressure is in communication with the second chamber.

14. An apparatus according to claim 13, wherein the independent source of negative pressure includes a control mechanism that allows periodic actuation thereof.

15. An apparatus according to claim 12, wherein the second chamber defines a sealed region.

16. An apparatus according to claim 12, wherein the second chamber defines an unsealed region.

17. An apparatus according to claim 11, wherein the displaceable member is selected from a flexible membrane, a piston, a diaphragm, and combinations thereof.

18. An apparatus according to claim 11, wherein the displaceable member provides resistance to withdrawal of air through the mouthpiece and automatically resumes a non-displaced orientation when air withdrawal is discontinued and air reenters the first chamber through the mouthpiece.

19. An apparatus according to claim 11, further including a plurality of mouthpieces that are interchangeably received by the first chamber.

20. An apparatus according to claim 11, further including a plurality of displaceable members that are interchangeably received with respect to the first chamber and are adapted to provide different resistance levels.
